(19) United States
(12) Patent Application Publication
(10) Pub. No.: US 2025/0217971 A1
(43) Pub. Date: Jul. 3, 2025

(54) METHOD AND DEVICE FOR CAMERA-BASED HEART RATE VARIABILITY MONITORING

(71) Applicant: Jing Wei CHIN, Hong Kong (HK)

(72) Inventors: Jing Wei CHIN, Hong Kong (HK); Tsz Tai CHAN, Hong Kong (HK)

(21) Appl. No.: 18/720,717

(22) PCT Filed: Sep. 15, 2023
(86) PCT No.: PCT/CN2023/119226

§ 371 Date: Jun. 17, 2024

Related U.S. Application Data
(60) Provisional application No. 63/407,305, filed on Sep. 16, 2022.

(51) Int. Cl. G06T 7/00; A61B 5/00; A61B 5/024; G06T 5/60; G06T 7/90; G06V 10/25; G06V 40/10; G06V 40/16

(52) U.S. Cl. CPC G06T 7/0012; A61B 5/02405; A61B 5/02416; A61B 5/7203; A61B 5/725; A61B 5/7267; G06T 5/60; G06T 7/90; G06V 10/25; G06V 40/15; G06V 40/161; A61B 2576/02; G06T 2207/10024; G06T 2207/30088; G06T 2207/30201

(57) ABSTRACT

A method for camera-based heart rate variability monitoring comprises: determining colored skin pixels in color image frames taken by a camera, and extracting mean RGB signal from the skin pixels (S100); obtaining rPPG signal from the mean RGB signal (S200); enhancing quality of the rPPG signal to obtain reconstructed rPPG signal by a windowing method (S300); detecting peaks of the reconstructed rPPG signal to obtain HRV values (S400); and analyzing peak to peak distances statistically to improve the HRV values (S500). A device for camera-based heart rate variability monitoring is also provided.

### RELATED APPLICATIONS

This application claims the priority of the provisional application named ‘METHOD FOR CAMERA-BASED HEART RATE VARIABILITY (HRV) MONITORING’ filed on 09.16.2022, with the application No. 63/407,305, which is hereby incorporated by reference in entirety.

### TECHNICAL FIELD

The present disclosure relates to a field of healthcare computer technology, and in particular to a method and device for camera-based heart rate variability monitoring.

### BACKGROUND

Human skin can be divided into three layers: epidermis, dermis and subcutaneous tissue. There are abundant capillaries in the dermis and subcutaneous tissue layers. The capillaries contain hemoglobin which can absorb light. The rush of blood caused by beating heart leads to periodic changes in the amount of hemoglobin in the capillaries, which in turn causes periodic changes in the amount of light absorbed by the skin. The periodic changes in the amount of light absorbed by the skin can still be captured by a camera, which can be used to monitor Heart Rate Variability (HRV).

### SUMMARY

According to various embodiments of the present disclosure, a method and device for camera-based heart rate variability monitoring is provided.

A method for camera-based heart rate variability monitoring, the method comprising:
determining colored skin pixels in color image frames taken by a camera, and extracting mean RGB signal from the skin pixels;
obtaining rPPG signal from the mean RGB signal;
enhancing quality of the rPPG signal to obtain reconstructed rPPG signal by a windowing method;
detecting peaks of the reconstructed rPPG signal to obtain HRV values; and
   analyzing peak to peak distances statistically to improve the HRV values.

A device for camera-based heart rate variability monitoring comprising:
a processor; and
a memory with computer code instructions stored thereon, the memory operatively coupled to the processor such that, when executed by the processor, the computer code instructions cause the computer system to implement:
determine colored skin pixels in color image frames taken by a camera, and extracting mean RGB signal from the skin pixels;
obtain rPPG signal from the mean RGB signal;
enhance quality of the rPPG signal to obtain reconstructed rPPG signal by a windowing method;
detect peaks of the reconstructed rPPG signal to obtain HRV values; and
analyze peak to peak distances statistically to improve the HRV values.

Details of one or more embodiments of the present disclosure will be given in the following description and attached drawings. Other features, objects and advantages of the present disclosure will become apparent from the description, drawings, and claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

In order to better describe and illustrate the embodiments and/or examples of the contents disclosed herein, reference may be made to one or more drawings. Additional details or examples used to describe the drawings should not be considered as limiting the scope of any of the disclosed contents, the currently described embodiments and/or examples, and the best mode of these contents currently understood.

FIG. 1 illustrates a flowchart of a method for camera-based heart rate variability monitoring according to an embodiment of the present disclosure;

FIG. 2 illustrates embodiments of cameras can be used in the method shown in FIG. 1;

FIG. 3 illustrates a flowchart of S100 in the method shown in FIG. 1 according to an embodiment of the present disclosure;

FIG. 4 illustrates a graph of a mean RGB signal according to an embodiment of the method shown in FIG. 1;

FIG. 5 illustrates a flowchart of S300 in the method shown in FIG. 1 according to an embodiment of the present disclosure;

FIG. 6 illustrates a window in an interpolated rPPG signal;

FIG. 7-A illustrates a flowchart of S320 in the method shown in FIG. 5 according to an embodiment of the present disclosure;

FIG. 7-B illustrates a flowchart of S320 in the method shown in FIG. 5 according to another embodiment of the present disclosure;

FIG. 8 illustrates coefficients of first order Scattering Transform according S322-B in FIG. 7-B;

FIG. 9 illustrates energy calculation according to S323-B in FIG. 7-B;

FIG. 10 illustrates adaptive band calculation according to S324-B in FIG. 7-B;

FIG. 11 illustrates a graph of a reconstructed rPPG signal according to an embodiment of the method shown in FIG. 5;

FIG. 12 illustrates a flowchart of S400 in the method shown in FIG. 1 according to an embodiment of the present disclosure;

FIG. 13 illustrates a flowchart of S500 in the method shown in FIG. 1 according to an embodiment of the present disclosure;

FIG. 14 illustrates a flowchart of S510 in the method shown in FIG. 13 according to an embodiment of the present disclosure;

FIG. 15 illustrates a SDNN graph according to an embodiment of the method shown in FIG. 13;

FIG. 16 illustrates a RMSSD graph according to an embodiment of the method shown in FIG. 13;

FIG. 17 illustrates a LF/HF graph according to an embodiment of the method shown in FIG. 13;

FIG. 18 illustrates a flowchart of a method for camera-based heart rate variability monitoring according to another embodiment of the present disclosure;

FIG. 19 illustrates a flowchart of S600 in the method shown in FIG. 18 according to an embodiment of the present disclosure;

FIG. 20 illustrates a diagram of a device for camera-based heart rate variability monitoring according to an embodiment of the present disclosure.

### DETAILED DESCRIPTION OF THE EMBODIMENTS

In order to facilitate the understanding of the present disclosure, the present disclosure will be described more fully below with reference to the relevant drawings. Preferred embodiments of the present disclosure are shown in the drawings. However, the present disclosure can be implemented in many different forms and is not limited to the embodiments described herein. On the contrary, the purpose of providing these embodiments is to make the disclosure of the present disclosure more thorough and comprehensive.

Unless otherwise defined, all technical and scientific terms used herein have the same meaning as commonly understood by one of ordinary skill in the art to which this invention belongs. The definitions are provided to aid in describing particular embodiments and are not intended to limit the claimed invention. The term “and/or” used herein includes any and all combinations of one or more related listed items.

In order to understand this application thoroughly, detailed steps and structures will be provided in the description below to explain the technical solution proposed by this application. Preferred embodiments of this application are described in detail below. However, in addition to these details, there may be other embodiments of this application.

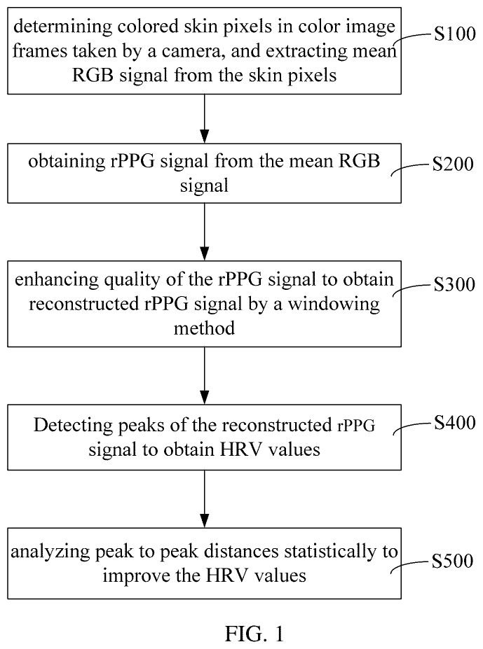

Referring to FIG. 1, a method for camera-based heart rate variability monitoring is provided. The method applies healthcare computer vision technology to extract health information from images. Heart Rate Variability (HRV) can be extracted from images on the use of health metrics and the health recommendations based on the health metrics. The method comprises the following steps:

S100, determining colored skin pixels in color image frames taken by a camera, and extracting mean RGB (Red, Green, Blue) signal from the skin pixels.

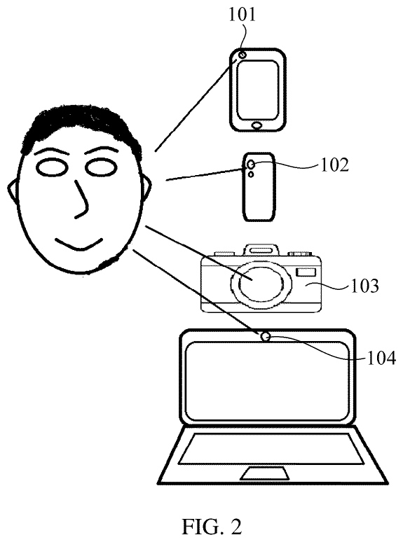

The method takes input in the form of color image frames that are taken from a camera. The color image frames can be a video. These color image frames can be captured using the color image capturing system which can be any camera. In some embodiments, the camera can be an independent camera or a built-in camera of a smart phone, tablet or laptop. Referring to FIG. 2, in an embodiment, the camera can be a front camera 101 of a smart phone, a rear camera 102 of a smart phone, an independent camera 103 or a webcam 104 of a laptop. The camera is used to record images of the user with timestamps.

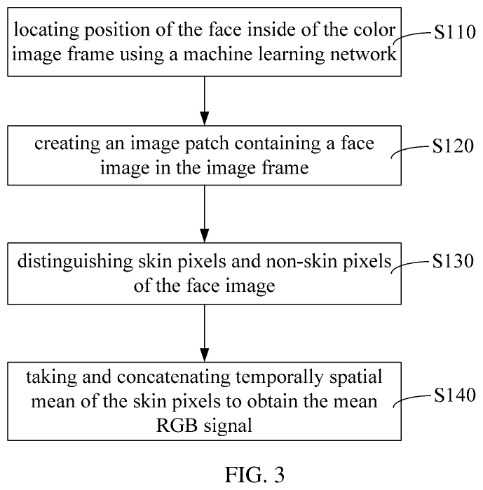

Referring to FIG. 3, in some embodiment, the step S100 comprises the following steps:

S110, locating position of the face inside of the color image frame using a machine learning network. A facial image capturing system can be utilized to extract images of user's face from the color image frame that have been taken and stored. This is achieved using a machine learning network. The color image frame captured by the camera is processed to locate the position of the face image inside of the color image frame.

S120, creating an image patch containing a face image in the image frame. The location of this face image is then referenced to create an image patch containing the full-face image of the user in the color image frame.

S130, distinguishing skin pixels and non-skin pixels of the face image. A skin detection algorithm is applied to face images to distinguish skin and non-skin parts of the face. In some embodiments, a region of interest (ROI) algorithm is applied to select some parts of the skin pixels for obtaining the mean RGB signal instead of the full face. After extracting regions of interest from the face image, those regions of interest can be tracked over an extended period of time. Regions of less interest can be removed, so efficiency can be improved with less computation. The monitoring accuracy can be improved.

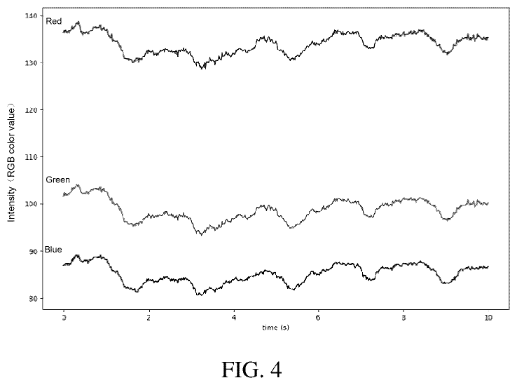

S140, taking and concatenating temporally spatial mean of the skin pixels to obtain the mean RGB signal. The spatial mean of the skin pixels is taken and concatenated temporally to obtain a signal which can be called the mean RGB signal, referring to FIG. 4.

S200, obtaining rPPG (remote photoplethysmography) signal from the mean RGB signal. In some embodiments, in the step of S200, a machine learning algorithm can be is applied to the mean RGB signal to obtain the rPPG signal to reduce noise. In an embodiment, machine learning or traditional algorithms such as POS (Plane Orthogonal to Skin) and CHROM can be applied to the mean RGB signal to obtain the rPPG signal.

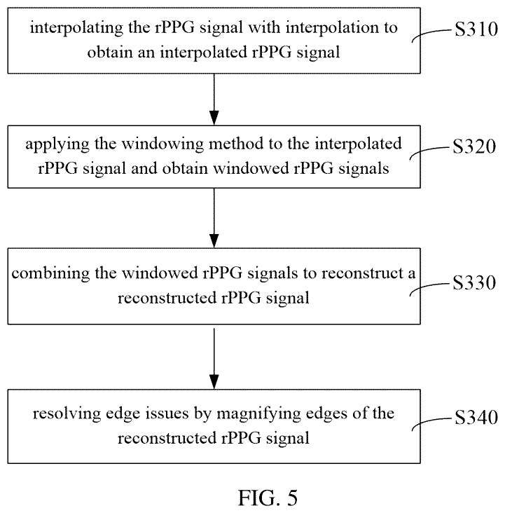

S300, enhancing quality of the rPPG signal to obtain reconstructed rPPG signal by a windowing method. In some embodiments, referring to FIG. 5, the step of S300 comprises the following steps:

S310, interpolating the rPPG signal with interpolation to obtain an interpolated rPPG signal. The rPPG signal can be interpolated with interpolation techniques such as cubic spline to the desired frequency such as 60 or 120 Hz. The step of S310 can be omitted if the timestamps of the video are not available.

S320, applying the windowing method to the interpolated rPPG signal and obtain windowed rPPG signals. The windowing method is applied to the interpolated rPPG signal. If the rPPG signal is not interpolated by S310, the interpolated rPPG signal can be the rPPG signal itself.

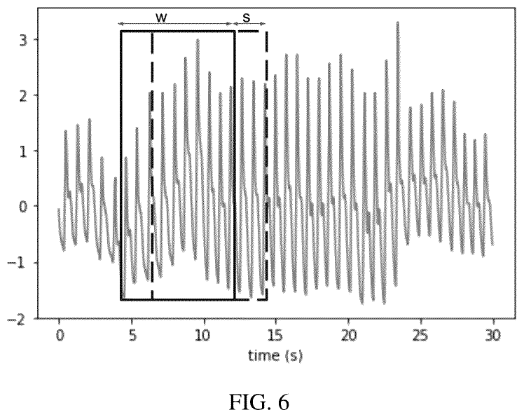

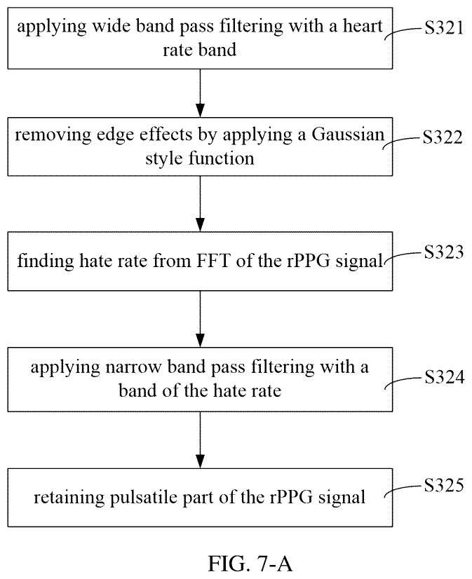

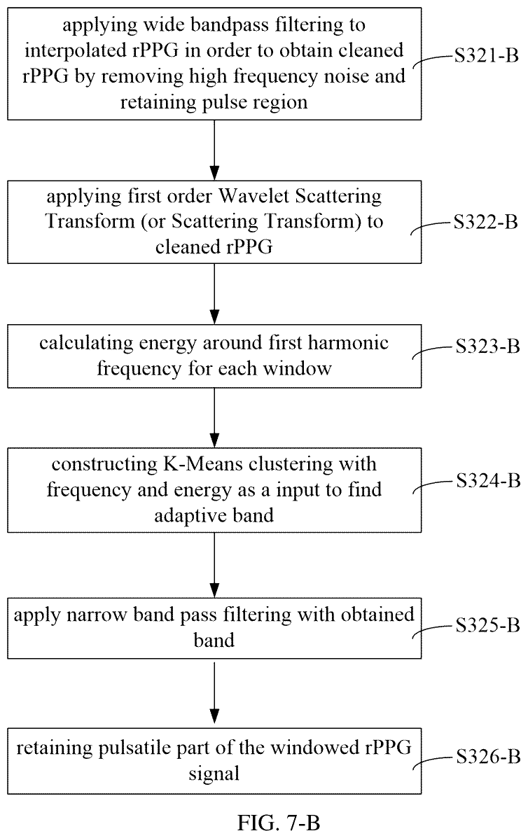

A window in an interpolated rPPG signal of the windowing step of S320 is shown in FIG. 6 for illustration purposes. For each segment of interpolated signal can be processed by the windowing step of S320, shown in details in FIG. 7:

S321, applying wide band pass filtering with a heart rate band such as 0.8-2.5 Hz. In this embodiment, band pass filtering such as Butterworth filter with wide HR (Heart Rate) band can be applied. The wide HR band can be 0.8-2.5 Hz. In other embodiments, the heart rate band can be wider than 0.8-2.5 Hz depending on the circumstances.

S322, removing edge effects by applying a Gaussian style function.

In the step of S322, a Gaussian style function such as the Hanning function can be applied to remove edge effects by.

S323, finding hate rate from FFT of the rPPG signal.

In the step of S323, the heart rate can be in units of Hz. The FFT is the abbreviation of the Fast Fourier Transform.

S324, applying narrow band pass filtering with a band of the hate rate. The band of the hate rate can be around heart rate frequency. The narrow band is narrower than the wide band.

S325, retaining pulsatile part of the windowed rPPG signal.

In this step, the mean of the signal from the signal itself can be subtracted to retain only the pulsatile part and remove the diffuse part.

In some embodiments, for each window over interpolated signals in the steps of S321 to S325, the size of each window is w and step size of each window is s.

Another embodiment of step S320 is shown in FIG. 7-B. According to this embodiment, the step of S320 can include the following steps:

S321-B, applying wide bandpass filtering to interpolated rPPG in order to obtain cleaned rPPG by removing high frequency noise and retaining pulse region. Examples of frequency range can be 0.7-5 Hz.

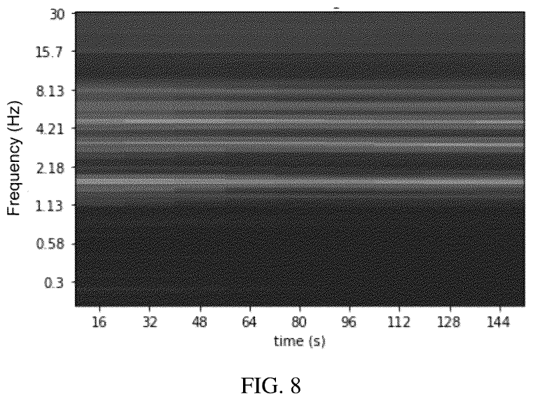

S322-B, applying first order Wavelet Scattering Transform (or Scattering Transform) to cleaned rPPG. FIG. 8 shows an example of first order Scattering Transform coefficients.

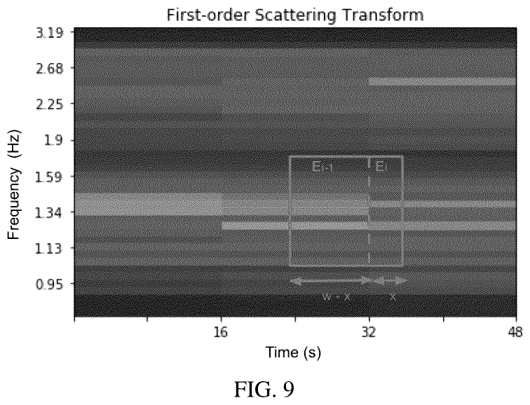

S323-B, calculating energy around first harmonic frequency for each window. Illustration of this step is given in FIG. 9.

In an embodiment, energy in this step could be calculated by the following formula:

$$E = \frac{w - x}{w} {E}_{i - 1} + \frac{x}{w} {E}_{i}$$

where w is window size, Ei is the energy at time i and x is difference between right-end of the window and time i. In other embodiment, the energy can be calculated by different formulas.

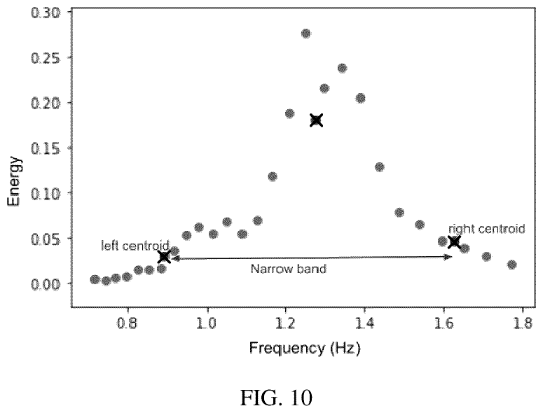

S324-B, constructing K-Means clustering with frequency and energy as an input to find adaptive band. FIG. 10 illustrates K (=3) Means clustering. Band-size in this case is [left centroid, right centroid].

S325-B, applying narrow band pass filtering with adaptive band which is obtained from step of S324-B.

S326-B, retaining pulsatile part of the windowed rPPG signal.

In this step, the mean of the signal from the signal itself can be subtracted to retain only the pulsatile part and remove the diffuse part.

In some embodiments, for each window over cleaned rPPG sigals in the steps of S321-B to S326-B, the size of each window is w and step size of each window is s.

S330, combining the windowed rPPG signals to reconstruct a reconstructed rPPG signal.

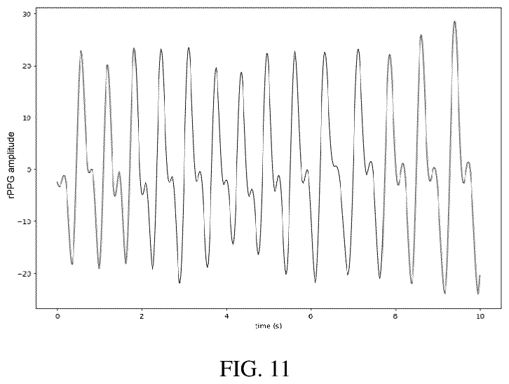

S340, resolving edge issues by magnifying edges of the reconstructed rPPG signal, referring to FIG. 11.

S400, detecting peaks of the reconstructed rPPG signal to obtain HRV values.

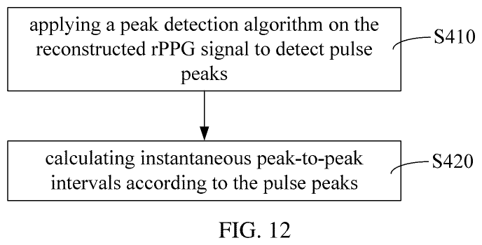

In some embodiments, referring to FIG. 12, the step of S400 can comprise the following steps:

S410, applying a peak detection algorithm on the reconstructed rPPG signal to detect pulse peaks.

S420, calculating instantaneous peak-to-peak intervals according to the pulse peaks. In an embodiment, the peak-to-peak intervals can include IBIs, RR intervals and NN intervals.

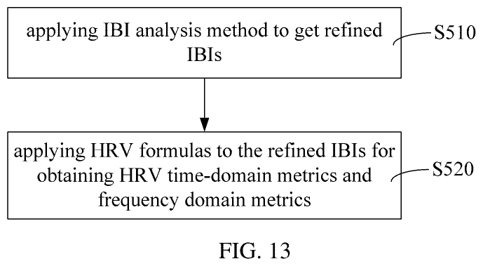

S500, analyzing peak to peak distances statistically to improve the HRV values. The step of S500 can reduce errors in HRV. Referring to FIG. 13, in some embodiments, the step of S500 comprises:

S510, applying IBI analysis method to get refined IBIs.

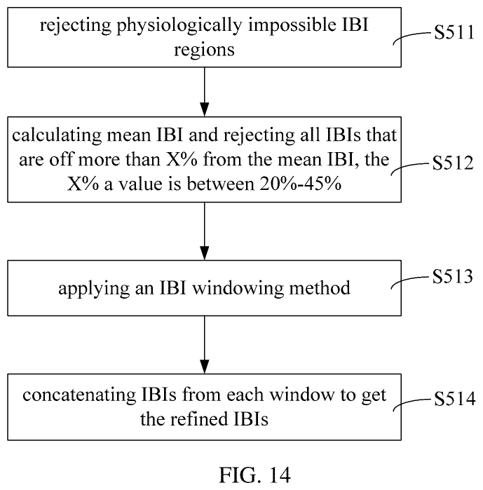

Referring to FIG. 14, the IBI analysis method can comprise the following steps:

S511, rejecting physiologically impossible IBI regions.

S512, calculating mean IBI and rejecting all IBIs that are off more than X % from the mean IBI, the X % a value is between 20%-45%.

S513, applying an IBI windowing method. In an embodiment, the IBI windowing method can comprises calculating mean IBI window, and rejecting IBIs that are off more than Y % from the mean IBI window, the Y % is a value between 10%-25%.

S514, concatenating IBIs from each window to get the refined IBIs.

Referring to FIG. 13, in some embodiments, the step of S500 can further comprise:

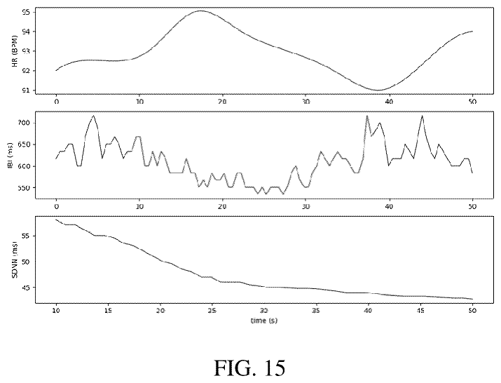

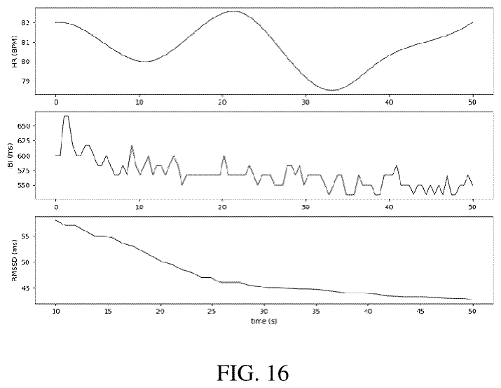

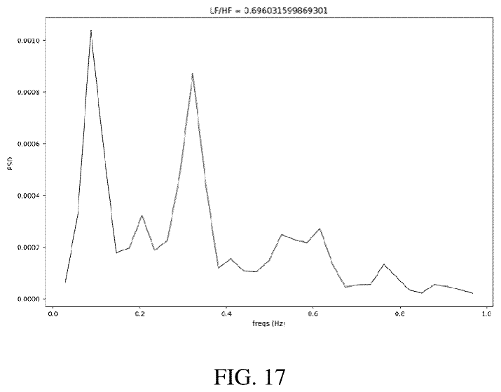

S520, applying HRV formulas to the refined IBIs for obtaining HRV time-domain metrics and frequency domain metrics. Referring to FIGS. 15-17, in an embodiment, the HRV time-domain metrics can include the standard deviation of normal to normal intervals (SDNN) and the root mean square of successive heartbeat interval differences (RMSSD), the frequency domain metrics can include low-frequency/high-frequency (LF/HF).

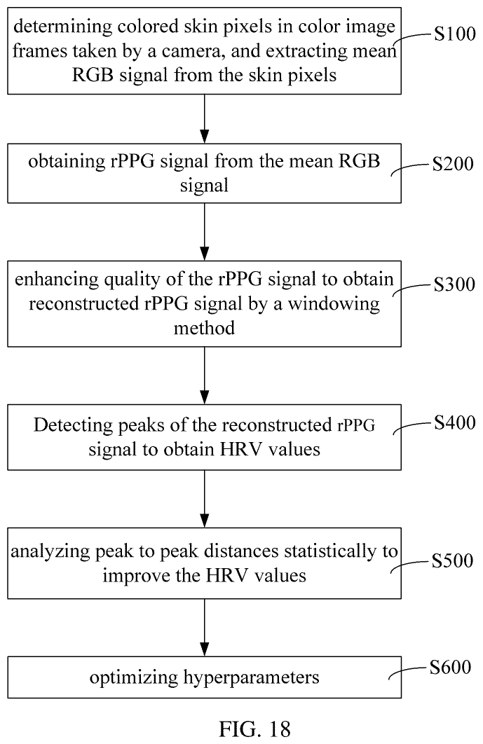

Referring to FIG. 18, in an embodiment, the method can further comprise:

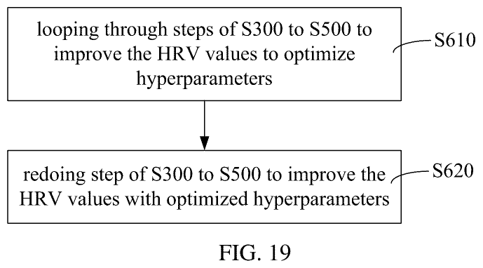

S600, optimizing hyperparameters. Referring to FIG. 19, the step of S600 comprises:

S610, looping through steps of S300 to S500 to improve the HRV values to optimize hyperparameters.

S620, redoing step of S300 to S500 to improve the HRV values with optimized hyperparameters obtained by S610.

Loop through steps of S300 to S500 to find optimal hyperparameters. Finally, after finding desired hyperparameters, redo steps of S300 to S500 with optimal hyperparameters and finish algorithm. The final result can be more accurate after Step of S600.

Overall, to be able to determine the Heart Rate Variability (HRV) information of a user, the method initially starts by capturing color image frames of the user. From the color image frames, computer vision techniques are then used to identify regions of interest that are then marked and tracked over a continuous period of time. These regions of interest are then fed into a series of algorithms that perform the extraction of the rPPG signal from the selected regions of interest. After that, the rPPG signal is cleaned and improved in quality by means of signal processing and statistical machine learning techniques. The cleaned signal is then used to estimate the HRV of the user which can be used to analyze the overall well-being and health of a user.

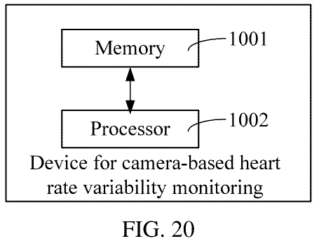

FIG. 20, a device for camera-based heart rate variability monitoring comprises a processor 1002 and a memory 1001 with computer code instructions stored thereon, the memory 1001 operatively coupled to the processor 1002 such that, when executed by the processor 1002, the computer code instructions cause the computer system to implement the steps of S100 to S500. In some embodiments, the computer code instructions also can cause the computer system to implement the step of S600.

The technical features in the foregoing embodiments may be randomly combined. For concise description, not all possible combinations of the technical features in the embodiment are described. However, provided that combinations of the technical features do not conflict with each other, the combinations of the technical features are considered as falling within the scope recorded in this specification.

The foregoing embodiments only describe several implementations of the disclosure, which are described specifically and in detail, and therefore cannot be construed as a limitation to the patent scope of the disclosure. It should be noted that, a person of ordinary skill in the art may further make variations and improvements without departing from the ideas of the disclosure, which all fall within the protection scope of the disclosure. Therefore, the protection scope of the disclosure is subject to the protection scope of the appended claims.

What is claimed is:

1. A method for camera-based heart rate variability monitoring, the method comprising:
determining colored skin pixels in color image frames taken by a camera, and extracting mean RGB signal from the skin pixels;
obtaining rPPG signal from the mean RGB signal;
enhancing quality of the rPPG signal to obtain reconstructed rPPG signal by a windowing method;
detecting peaks of the reconstructed rPPG signal to obtain HRV values; and
analyzing peak to peak distances statistically to improve the HRV values.

2. The method as in claim 1, wherein step of determining colored skin pixels in color image frames taken by a camera, and extracting mean RGB signal from the skin pixels, comprises:
locating position of the face inside of the color image frame using a machine learning network;
creating an image patch containing a face image in the image frame;
distinguishing skin pixels and non-skin pixels of the face image; and
taking and concatenating temporally spatial mean of the skin pixels to obtain the mean RGB signal.

3. The method as in claim 2, wherein in step of taking and concatenating temporally spatial mean of the skin pixels to obtain the mean RGB signal, a region of interest algorithm is applied to select some parts of the skin pixels for obtaining the mean RGB signal.

4. The method as in claim 1, wherein in step of obtaining rPPG signal from the mean RGB signal, a machine learning algorithm is applied to the mean RGB signal to obtain the rPPG signal.

5. The method as in claim 1, wherein step of enhancing quality of the rPPG signal to obtain reconstructed rPPG signal by a windowing method, comprising:
interpolating the rPPG signal with interpolation to obtain an interpolated rPPG signal;
applying the windowing method to the interpolated rPPG signal and obtain windowed rPPG signals;
combining the windowed rPPG signals to reconstruct a reconstructed rPPG signal; and
resolving edge issues by magnifying edges of the reconstructed rPPG signal.

6. The method as in claim 1, wherein in step of enhancing quality of the rPPG signal to obtain reconstructed rPPG signal by a windowing method, the windowing method comprises:
applying wide band pass filtering with a heart rate band;
removing edge effects by applying a Gaussian style function;
finding hate rate from FFT of the rPPG signal;
applying narrow band pass filtering with a band of the hate rate; and
retaining pulsatile part of the rPPG signal.

7. The method as in claim 6, wherein the heart rate band is 0.8-2.5 Hz.

8. The method as in claim 1, wherein in step of enhancing quality of the rPPG signal to obtain reconstructed rPPG signal by a windowing method, the windowing method comprises:
applying wide bandpass filtering to interpolated rPPG in order to obtain cleaned rPPG by removing high frequency noise and retaining pulse region;
applying first order Wavelet Scattering Transform or Scattering Transform to the cleaned rPPG;
calculating energy around first harmonic frequency for each window;
constructing K-Means clustering with frequency and energy as an input to find adaptive band;
applying narrow band pass filtering with the adaptive band; and
retaining pulsatile part of windowed rPPG signal.

9. The method as in claim 1, wherein step of detecting peaks of the reconstructed rPPG signal to obtain HRV values, comprising:
applying a peak detection algorithm on the reconstructed rPPG signal to detect pulse peaks; and
calculating instantaneous peak-to-peak intervals according to the pulse peaks.

10. The method as in claim 9, wherein the peak-to-peak intervals include IBIs, RR intervals and NN intervals.

11. The method as in claim 1, wherein step of analyzing peak to peak distances statistically to improve the HRV values, comprising:
applying IBI analysis method to get refined IBIs.

12. The method as in claim 11, wherein the IBI analysis method comprises:
rejecting physiologically impossible IBI regions;
calculating mean IBI and rejecting all IBIs that are off more than X % from the mean IBI, the X % a value is between 20%-45%;
applying an IBI windowing method; and
concatenating IBIs from each window to get the refined IBIs.

13. The method as in claim 12 wherein the IBI windowing method comprises:
calculating mean IBI window; and
rejecting IBIs that are off more than Y % from the mean IBI window, the Y % is a value between 10%-25%.

14. The method as in claim 11, wherein step of analyzing peak to peak distances statistically to improve the HRV values, further comprising:
applying HRV formulas to the refined IBIs for obtaining HRV time-domain metrics and frequency domain metrics.

15. The method as in claim 14, wherein the HRV time-domain metrics includes SDNN and RMSSD; the frequency domain metrics includes LF/HF.

16. The method as in claim 1, wherein the method further comprising:
looping through step of enhancing quality of the rPPG signal to obtain reconstructed rPPG signal by a windowing method to step of analyzing peak to peak distances statistically to improve the HRV values to optimize hyperparameters; and
redoing step of enhancing quality of the rPPG signal to obtain reconstructed rPPG signal by a windowing method to step of analyzing peak to peak distances statistically to improve the HRV values with optimized hyperparameters.

17. The method as in claim 1, wherein in step of determining colored skin pixels in color image frames taken by a camera, and extracting mean RGB signal from the skin pixels, the camera is an independent camera or a built-in camera of a smart phone, tablet or laptop.

18. A device for camera-based heart rate variability monitoring comprising:
a processor; and
a memory with computer code instructions stored thereon, the memory operatively coupled to the processor such that, when executed by the processor, the computer code instructions cause the computer system to implement:
determine colored skin pixels in color image frames taken by a camera, and extracting mean RGB signal from the skin pixels;
obtain rPPG signal from the mean RGB signal;
enhance quality of the rPPG signal to obtain reconstructed rPPG signal by a windowing method;
detect peaks of the reconstructed rPPG signal to obtain HRV values; and
analyze peak to peak distances statistically to improve the HRV values.
